(19) United States
(12) Patent Application Publication
(10) Pub. No.: US 2022/0017928 A1
(43) Pub. Date: Jan. 20, 2022

(54) PRETREATMENT OF SOFTWOOD

(71) Applicant: IOGEN CORPORATION, Ottawa (CA)

(72) Inventors: Joshua MARLEAU-GILLETTE, Gloucester (CA); Daniel G. MACDONALD, Orleans (CA); Jeffrey S. TOLAN, Ottawa (CA); Brian FOODY, Ottawa (CA)

(21) Appl. No.: 17/309,080

(22) PCT Filed: Nov. 9, 2018
(86) PCT No.: PCT/CA2018/000216

§ 371 Date: Apr. 21, 2021

(51) Int. Cl. C12P 7/10; C12P 19/02; C12P 19/14; C10L 1/02; C12P 5/02; C12P 11/00

(52) U.S. Cl. CPC C12P 7/10; C12P 19/02; C12P 19/14; C10L 1/02; C10L 2200/0469; C12P 11/00; C10L 2290/26; C10L 2290/06; C12P 2201/00; C12P 5/023

(57) ABSTRACT

A process for producing a fuel from a softwood. A feedstock containing softwood is pretreated, where the pretreating includes heating the feedstock in a pretreatment liquor containing sulfur dioxide and bisulfite salt. The heating is conducted between 110° C. and 160° C. The pretreatment liquor has a sulfur dioxide concentration that is greater than 6.5 wt % on liquor and a pH at 25° C. that is less than 1.3. The cellulose in the pretreated material is hydrolysed to glucose. The glucose may be fermented to a fermentation product such as ethanol.

### CROSS REFERENCE TO RELATED APPLICATIONS

Not applicable.

### TECHNICAL FIELD

The present disclosure relates generally to a process and/or system for pretreating softwood, and in particular, to a process and/or system for converting softwood to glucose or a biofuel, where the softwood is subject to a pretreatment with sulfur dioxide and/or bisulfite prior to enzymatic hydrolysis.

### BACKGROUND

Softwood may be an important feedstock in the bioconversion of lignocellulosic biomass to biofuels. Softwood is the primary source of lignocellulosic biomass in many areas of the northern hemisphere and can be obtained sustainably. Unfortunately, softwood is generally considered to be one of the most difficult lignocellulosic feedstock to enzymatically hydrolyze to sugars.

Relative to hardwood and herbaceous crops, softwood generally has a higher lignin content. Lignin-derived inhibition can be a major obstacle in the enzymatic hydrolysis of softwood. In addition, the lignin and/or hemicellulose components in softwood may differ significantly from that in hardwood and/or herbaceous crops. For example, the hemicellulose in softwood may be largely made up of mannose, which is a hexose that can be fermented by normal Baker's yeast, whereas the hemicellulose in hardwood and agricultural residues may be largely made up of xylose. Furthermore, the content of acetylated groups in softwood hemicellulose may not be as high as in hardwood or herbaceous hemicellulose. The presence of acetylated groups may promote autohydrolysis. Accordingly, processes developed for the bioconversion of agricultural residues and/or hardwood to sugars are not necessarily ideal for softwood.

Various processes for pretreating softwood prior to enzymatic hydrolysis have been proposed, including dilute acid, sulfur dioxide (SO₂)-catalyzed steam explosion, organosolv, and sulfite-pulping based pretreatments. Sulfite pulping, which can produce wood pulp by removing lignin from wood chips, may be categorized as: (a) acid sulfite (e.g., pH 1-2); (b) bisulfite (e.g., pH 2-6); (c) neutral sulfite (e.g., pH 6-9⁺); or (d) alkaline sulfite (e.g., pH 10⁺) pulping. In acid sulfite pulping, wherein the cooking liquor has relatively high free SO₂ content, relatively low temperatures (e.g., 130° C. to 145° C.) and long heat-up times (e.g., 6 hours) are used to allow homogeneous distribution of active cooking chemicals into the wood chips and/or to prevent lignin condensation, which can result in a “black cook.”

Even with low temperatures and relatively long heat-up times, lignin condensation can be problematic when acid sulfite processes are used to pulp softwoods, which can have high resin content. For example, at low pH values (e.g., below 1.5), the presence of resinous extractives (e.g., phenolic compounds) can favour the condensation of lignin over sulfonation reactions, which prevents efficient delignification. In particular, the heartwood of pine can contain relatively high amounts of phenolic compounds such as pinosylvin, which may condense with lignin moieties. The extent of lignin condensation can be reduced by cooking at higher pH values, which tends to favour the sulfonation of lignin over the condensation reactions.

One sulfite pulping based pretreatment that has been proposed for treating softwood is the Sulfite Pretreatment to Overcome the Recalcitrace of Lignocelluloses (SPORL) process. Although based on the sulfite pulping of wood, SPORL has been reported to differ from sulfite pulping in that it uses shorter reaction times, a slightly higher temperature, and often a lower sulfite loading. However, as in dilute acid pretreatment or SO₂-catalyzed steam explosion pretreatment, lignin dissolution may be limited in SPORL pretreatments.

The use of sulfite in SPORL is believed to increase the pH value (e.g., relative to dilute acid pretreatment) and prevent extensive condensation of the lignin. When applied to softwood, and particularly when using: (a) SO₂ rather than H₂SO₄, (b) pretreatment temperatures less than 160° C., and (c) pretreatment times greater than about 30 minutes, SPORL experiments have relied on providing sufficient sulfite to increase the pH to values of about 1.4 or higher.

### SUMMARY

According to one aspect of the invention there is provided a process for producing a fuel from softwood, said process comprising: (a) obtaining a feedstock comprising softwood; (b) pretreating the feedstock, said pretreating comprising heating the feedstock in a pretreatment liquor comprising sulfur dioxide and bisulfite salt, said heating conducted between 110° C. and 160° C., wherein the pretreatment liquor has a pH at 25° C. that is less than 1.3 and has a sulfur dioxide concentration that is greater than 6.5 wt % on liquor, (c) obtaining a slurry of pretreated material produced in (a), said slurry having a solid fraction comprising cellulose and a liquid fraction comprising solubilized hemicellulose; (d) hydrolyzing the cellulose to glucose, said hydrolyzing comprising adding cellulase to at least the solid fraction; (e) fermenting the glucose to a fermentation product, said fermenting comprising adding a microorganism to at least the glucose; and (f) recovering the fermentation product, wherein the fuel comprises the fermentation product.

According to one aspect of the invention there is provided a process for producing ethanol comprising: (a) obtaining a feedstock, said feedstock comprising softwood woodchips; (b) pretreating the feedstock, said pretreating comprising heating the feedstock in a pretreatment liquor comprising sulfur dioxide and bisulfite salt, said heating conducted between 110° C. and 160° C. for at least 30 minutes, wherein the pretreatment liquor has a pH at 25° C. that is less than 1.3 and has a sulfur dioxide concentration that is greater than 6.5 wt % on liquor; (c) obtaining a slurry of pretreated material produced in (a), said slurry having a solid fraction comprising cellulose and a liquid fraction comprising solubilized hemicellulose; (d) hydrolyzing the cellulose to glucose, said hydrolyzing comprising adding cellulase to at least the solid fraction; (e) fermenting the glucose to ethanol, said fermenting comprising adding a microorganism to at least the glucose; (f) recovering the ethanol; and (g) producing one or more products from the liquid fraction, said one or more products comprising at least one of xylose, xylitol, methane, ethanol, or lignosulfonate.

### BRIEF DESCRIPTION OF THE DRAWINGS

FIG. 1 is a plot of cellulose conversion versus time for the enzymatic hydrolysis of washed solids obtained from pretreatment of red pine in a pretreatment liquor containing SO₂ and bisulfite salt, at 140° C., where the SO₂ concentration was 8.4 wt % (on liquor) and the pretreatment time was 2 hours;

FIG. 2 is a plot of cellulose conversion versus time for the enzymatic hydrolysis of washed solids obtained from pretreatment of red pine in a pretreatment liquor containing SO₂ and bisulfite salt, at 140° C., where the SO₂ concentration was 8.4 wt % (on liquor) and the pretreatment time was 3 hours; and

FIG. 3 is a plot of cellulose conversion versus time for the enzymatic hydrolysis of washed solids obtained from pretreatment of red pine in a pretreatment liquor containing SO₂ and bisulfite salt, at 140° C., where the SO₂ concentration was 11.1 wt % (on liquor) and the pretreatment time was 3 hours.

### DETAILED DESCRIPTION

Certain exemplary embodiments of the invention now will be described in more detail, with reference to the drawings, in which like features are identified by like reference numerals. The invention may, however, be embodied in many different forms and should not be construed as limited to the embodiments set forth herein.

The terminology used herein is for the purpose of describing certain embodiments only and is not intended to be limiting of the invention. For example, as used herein, the singular forms “a”, “an,” and “the” may include plural references unless the context clearly dictates otherwise. The terms “comprises”, “comprising”, “including”, and/or “includes”, as used herein, are intended to mean “including but not limited to”. The term “and/or”, as used herein, is intended to refer to either or both of the elements so conjoined. The phrase “at least one” in reference to a list of one or more elements, is intended to refer to at least one element selected from any one or more of the elements in the list of elements, but not necessarily including at least one of each and every element specifically listed within the list of elements. Thus, as a non-limiting example, the phrase “at least one of A and B” may refer to at least one A with no B present, at least one B with no A present, or at least one A and at least one B in combination. In the context of describing the combining of components by the “addition” or “adding” of one component to another, those skilled in the art will understand that the order of addition is not critical (unless stated otherwise). The terms “first”, “second”, etc., may be used to distinguish one element from another, and these elements should not be limited by these terms. Unless defined otherwise, all technical and scientific terms used herein have the same meanings as commonly understood by one of ordinary skill in the art.

The instant disclosure describes an improved method of converting softwood to biofuels. More specifically, the instant disclosure describes a process that includes pretreating a feedstock including softwood at a temperature between 110° C. and 160° C., using a pretreatment liquor containing SO₂, and preferably a bisulfite salt, wherein the pH of the pretreatment liquor is below about 1.3 (measured at ambient temperature) near the start of the pretreatment. Advantageously, it has been found that by adjusting the amount of SO₂, bisulfite salt, pretreatment time, and pretreatment temperature, as required, improved hydrolysis results can be obtained for softwood at these relatively low pH values. For example, in one embodiment, the concentration of SO₂ in the pretreatment liquor is greater than about 6.5 wt % (expressed as weight percent SO₂, based on weight of the pretreatment liquor). The relatively high SO₂ concentration promotes sulfonation, and thus lignin dissolution. The low pH values contribute to hemicellulose dissolution, which can improve the enzymatic hydrolysis. In accordance with one embodiment, the cellulose in the pretreated softwood is hydrolyzed to glucose with enzymes. In one embodiment, the glucose is fermented to a fermentation product, such as ethanol.

### Feedstock

In one embodiment, the feedstock includes softwood (coniferous wood). Some examples of softwood include cedar, fir, pine, spruce, hemlock, cypress, larch, and yew. In one embodiment, the feedstock includes softwood sapwood, softwood heartwood, softwood bark, or any combination thereof. In one embodiment, the feedstock includes the sapwood and/or heartwood of softwood. In one embodiment, the feedstock includes softwood trimmings, or slash. For example, in one embodiment, the feedstock contains otherwise unwanted branches, tops, and/or stumps, of softwood, produced during logging operations. In one embodiment, the feedstock includes softwood mixed with another type of lignocellulosic biomass (e.g., hardwood or herbaceous). In one embodiment, the feedstock includes softwood bark. In one embodiment, the feedstock does not include softwood bark. In one embodiment, the feedstock includes softwood killed by insects. In one embodiment, the feedstock includes pine killed by insects (e.g., mountain pine beetle).

In one embodiment, the feedstock includes softwood selected from Cedar (e.g., Juniperus virginiana, Thuja plicata, Thuja occidentalis), Cypress (e.g. Chamaecyparis, Cupressus Taxodium), Douglas Fir (Pseudotsuga menziesii), Fir (e.g. Abies balsamea, Abies alba), Hemlock (e.g. Tsuga canadensis, Tsuga mertensiana, Tsuga heterophylla); Larch (e.g., Larix laricina, Larix occidentalis), Pine (e.g. Pinus resinosa, Pinus nigra, Pinus strobus, Pinus banksiana, Pinus taeda, Pinus contorta, Pinus palustris, Pinus rigida, Pinus ponderosa, Pinus radiata, Pinus sylvestris, Pinus echinata, Pinus elliotti, Pinus lambertiana, Pinus monticola, Pinus virginiana), Redwood, Spruce (e.g. Picea abies, Picea mariana), and combinations and/or hybrids thereof.

In one embodiment, the feedstock comprises resinous softwood. Resinous softwood is softwood that has a relatively high resin content. For example, Douglas fir and pines are generally considered to be resinous, whereas spruce is generally not considered resinous softwood. In one embodiment, the feedstock includes heartwood, sapwood, and/or bark from resinous softwood.

In one embodiment, the feedstock includes Douglas Fir or pine. In one embodiment, the feedstock includes Red or Norway Pine (Pinus resinosa), Austrian or Black or Corsican pine (Pinus nigra), Eastern White Pine (Pinus strobus), Jack Pine (Pinus banksiana), Loblolly Pine (Pinus taeda), Lodgepole Pine (Pinus contorta), Longleaf Pine (Pinus palustris), Pitch Pine (Pinus rigida), Ponderosa Pine (Pinus ponderosa), Monterey or Radiata Pine (Pinus radiata), Scots Pine (Pinus sylvestris), Shortleaf Pine (Pinus echinata), Slash Pine (Pinus elliotti), Sugar Pine (Pinus lambertiana), Western White Pine (Pinus monticola) Virginia or Scrub Pine (Pinus virginiana), or any combination thereof.

In general, the softwood feedstock may be of any age (e.g., fresh or conditioned) and of any moisture content. For example, the softwood may be stored for a certain time period, inside or outside, and/or may be wet or dry.

### Feedstock Preparation

In one embodiment, the feedstock fed into pretreatment includes softwood that has been subject to one or more mechanical processes that cuts and/or otherwise breaks up the softwood (e.g., the mechanical process may use shear or impact mechanisms).

In one embodiment, the feedstock is received as woodchips, wood shavings, wood pellets, sawdust, wood powder, or any combination thereof. For example, in one embodiment, the process includes collecting sawdust and/or wood shavings from a sawmill or lumber mill. In one embodiment, the process includes obtaining hog fuel, pin chips, and/or other by-products produced by a sawmill as a feedstock to the process.

In one embodiment, softwood is received as trees, logs, wood blocks, and/or slash and is subject to one or more mechanical processes that produces woodchips, wood shavings, wood pellets, sawdust, wood powder, or any combination thereof. For example, in one embodiment, the process includes feeding softwood (e.g., large logs, blocks, short rotational trees, slash, etc.) into a wood chipper. Wood chippers are often used to cut wood for pulp, mulch, and/or other wood products (e.g., using disks or knives). In one embodiment, the process includes feeding softwood into a hammer mill.

In one embodiment, the feedstock includes or primarily contains woodchips. In general, woodchips may be spherical, cubical, rectangular, cone, or irregularly shaped. For example, woodchips may have chiseled or angled ends. Using woodchips is advantageous in that they are relatively easy to convey and/or feed into the pretreatment reactor and/or because, when sized appropriately, they do not clog screens (e.g., used in a digester).

In general, woodchips may come in various lengths, widths, and thicknesses. In one embodiment, the feedstock includes woodchips that are between about 5 mm and about 50 mm long, between about 5 mm and 50 mm wide, and between about 2 mm and about 12 mm thick. In one embodiment, the feedstock includes woodchips that are between about 10 mm and about 30 mm long, between about 10 mm and 50 mm wide, and between about 2 mm and about 10 mm thick. In one embodiment, the feedstock includes woodchips that are between about 10 mm and about 30 mm long, between about 10 mm to 50 mm wide, and between about 2 mm and about 8 mm thick. In one embodiment, the feedstock includes woodchips that are between about 10 mm and about 30 mm long, between about 10 mm and 50 mm wide, and between about 3 mm and about 12 mm thick. In one embodiment, the feedstock includes woodchips that are between about 12 mm and about 25 mm long and between about 2 mm and about 10 mm thick.

In one embodiment, the feedstock includes woodchips that have an average length that is less than 4 cm, less than 3 cm, less than 2 cm, less than 1.5 cm, less than 1.25 cm, less than 1 cm, less than 0.8 cm, less than 0.7 cm, less than 0.6 cm, or less than 0.5 cm.

In one embodiment, the feedstock includes woodchips that have an average thickness that is less than 3 cm, less than 2 cm, less than 1.5 cm, less than 1.25 cm, less than 1 cm, less than 0.8 cm, or less than 0.6 cm. In one embodiment, the feedstock includes woodchips having an average thickness between about 1 mm and about 1.5 cm, between about 2 mm and about 1 cm, between about 2 mm and about 9 mm, between about 3 mm and about 8 mm, between about 4 mm and about 8 mm, between about 5 mm and about 8 mm, or between about 7 mm and about 8 mm. For example, in one embodiment the feedstock includes softwood chips having an average thickness between about 2 mm and about 8 mm. The geometric properties of woodchips may be measured (e.g., during the process) using any known methods (e.g., optical metering).

In one embodiment, the feedstock includes or primarily contains sawdust.

Sawdust or “wood dust” includes by-products or waste products from woodworking operations such as sawing, milling, planing, routing, drilling, and sanding. Woodchips having a thickness less than about 3 mm-5 mm may be also known as sawdust. Wood powder may be produced when wood is crushed and/or pulverized into a powder or fine particles (e.g., using a ball mill).

In one embodiment, the feedstock is produced by subjecting softwood to one or more mechanical processes that provide size reduction. For example, in one embodiment, the mechanical process(es) include chipping, sawing, chopping, shredding, agitation, grinding, compression, refining, and/or milling. In one embodiment, the softwood is fed to a mobile chipper, a vertical feeding chipper, a horizontal feeding chipper, a drum chipper, a disk chipper, or any combination thereof, to produce woodchips and/or sawdust.

In one embodiment, where the feedstock includes woodchips or sawdust, the feedstock is subject to a size sorting process. Size sorting may be conducted in order to provide a relatively uniform chip/particle size and/or to reduce chip/particle size distribution. In one embodiment, the feedstock is woodchips and is subject to a size sorting by passing it over a series of screens to partition the woodchips into different sizes (e.g., fines, accepts, or oversized pieces). Wood pieces that do not pass through the screen(s) may then be subject to further mechanical processing (e.g., fed to a re-chipper or slicer). In one embodiment, the feedstock includes woodchips and is passed through one or more screens in order to provide woodchips having a predetermined maximum width and/or thickness.

In one embodiment, the feedstock includes woodchips and is passed through/over one or more screens. In one embodiment, the feedstock includes softwood woodchips that have a width and/or thickness that is less than about 3 cm, less than about 2 cm, less than about 1.5 cm, less than about 1.25 cm, less than about 1 cm, less than about 0.8 cm, or less than about 0.6 cm. In one embodiment, the feedstock includes softwood woodchips that have a width and/or thickness that is between about 2 mm and about 9 mm. In one embodiment, the feedstock includes softwood woodchips that have a width and/or thickness that is between about 2 mm and about 8 mm. In one embodiment, the feedstock includes softwood woodchips that have a width and/or thickness that is between about 3 mm and about 8 mm. In one embodiment, the feedstock includes sawdust and is passed through a mesh screen (e.g., up to 20 Tyler Mesh).

In one embodiment, the feedstock includes conditioned softwood. Conditioning, which weakens bark and foliage and their bond to wood, may be accomplished by storing the wood and/or exposing the wood to steam. Conditioning may be conducted before or after size reduction. For example, conditioning may be accomplished by, for example, storing woodchips in a pile for about 6 weeks, or may be accomplished by a short exposure to steam (e.g., 10 minutes).

In one embodiment, the feedstock includes debarked softwood. Bark, which may be a contaminant and/or undesirable during the pretreatment, may be removed using abrasion. Debarking may be conducted on softwood logs, softwood blocks, and/or on mechanically processed softwood. For example, many debarkers are designed to remove bark from logs or stems (trees) prior to sawing and/or chipping. Some examples of log debarkers include drum, ring, Rosser head, and flail debarkers. In one embodiment, debarking is conducted by agitating conditioned woodchips vigorously (e.g., in water). In this embodiment, segregation of the bark and wood components (e.g., heartwood and sapwood) from the woodchips may be achieved by screening.

In one embodiment, the feedstock includes woodchips, wood shavings, and/or sawdust from fresh or conditioned softwood. In one embodiment, the feedstock includes rejects from a pulp and paper process. For example, in one embodiment, the feedstock includes wood chips that are not expected to produce suitable qualities of pulp and paper. In one embodiment, the feedstock includes pulp screening rejects (e.g. chips that were not fiberized properly). In one embodiment, the feedstock includes pulp knotters rejects.

In one embodiment, the feedstock is washed, deiced, leached, soaked, or pre-steamed. Washing, which may be performed before, during, or after size reduction, may remove sand, grit, and/or fine particles from the feedstock. In one embodiment, the softwood logs are subject to a deicing and/or washing step prior to debarking and/or size reduction. Soaking woodchips may allow water and/or allow pretreatment chemical(s) to more uniformly impregnate the feedstock, which in turn may provide even cooking in the heating step of pretreatment. In general, soaking may be carried out at any suitable temperature (e.g., below 100° C.) and/or for any suitable duration. In one embodiment, the feedstock is pre-steamed.

In one embodiment, the feedstock is slurried (e.g., in water) in order to facilitate pumping of the feedstock. In one embodiment, the feedstock is not slurried, and is moved using a conveyer (e.g., a belt conveyer or pneumatic conveyer).

In embodiments where the feedstock is washed, leached, soaked, pre-steamed, or slurried, excess water may be removed prior to adding the pretreatment liquor. Pre-steaming may improve packing and/or remove air. In one embodiment, the condensate provided by pre-steaming is drained from the feedstock prior to entering the pretreatment reactor and/or within the pretreatment reactor. At least partially dewatering (e.g., at least some water is removed) the feedstock may provide a specific consistency.

### Pretreatment

The term “pretreating” or “pretreatment”, as used herein, refers to one or more steps wherein the feedstock is treated to improve the enzymatic digestibility thereof. For example, in one embodiment, the pretreatment disrupts the structure of the feedstock material such that the cellulose therein is more susceptible and/or accessible to enzymes in a subsequent enzymatic hydrolysis of the cellulose.

In one embodiment, the pretreatment conditions are selected to improve the enzymatic digestibility of the feedstock, thereby increasing the glucose yield and/or increasing the rate of hydrolysis (for a given yield). In one embodiment, pretreating the feedstock allows at least 50 wt %, at least 60 wt %, at least 70 wt %, at least 80 wt %, or at least 90 wt % of the cellulose in the feedstock to be converted to glucose (based on the cellulose available in the feedstock).

In one embodiment, the pretreatment conditions are selected to improve both the glucose yield from the cellulose fraction, and the product yield from the hemicellulose fraction. Hemicellulose, which is present along with cellulose in most plant cell walls, is a heterogeneous polymer that may contain pentose (e.g., xylose and arabinose) and hexose (e.g., mannose, glucose and galactose) units. Some examples of hemicelluloses include xylan, arabinoxylan, and glucomannan. In hardwood, the main hemicellulose is often xylan, whereas in softwood, the main hemicellulose is often glucomannan.

In one embodiment, the pretreatment includes heating the softwood (e.g., wood chips, wood shavings, sawdust, and/or powder) at an elevated temperature in an aqueous pretreatment liquor containing sulfur dioxide (SO₂). In one embodiment, the aqueous pretreatment liquor contains both SO₂ and a bisulfate salt (e.g., salt of HSO₃⁻), which may for example, have a Na⁺, Ca²⁺, K⁺, Mg²⁺, or NH₄⁺ counter ion.

In one embodiment, the pretreatment includes heating the softwood in the aqueous pretreatment liquor within the temperature range from about 110° C. to about 160° C. In one embodiment, the pretreatment is conducted between about 110° C. and about 150° C., between about 120° C. and about 150° C., between about 120° C. and about 145° C., between about 125° C. and about 145° C., or between about 130° C. and about 140° C. In one embodiment, the pretreatment is conducted at about 130° C., about 135° C., or about 140° C. Using pretreatment temperatures between about 110° C. and about 150° C. advantageously avoids the equipment and/or hemicellulose degradation associated with pretreatments at relatively high temperatures (e.g., greater than 160° C.).

In one embodiment, the pretreatment includes heating the softwood in the aqueous pretreatment liquor within the temperature range from about 110° C. to about 160° C. for at least 30 minutes. In one embodiment, the pretreatment is conducted at a temperature(s) between about 110° C. and about 160° C. for at least 60 minutes, at least 80 minutes, at least 90 minutes, at least 100 minutes, at least 120 minutes, at least 140 minutes, at least 160 minutes, at least 180 minutes, at least 200 minutes, at least 220 minutes, or about 240 minutes. In one embodiment, the pretreatment is conducted at a temperature(s) between about 120° C. and about 150° C. for at least 60 minutes, at least 80 minutes, at least 90 minutes, at least 100 minutes, at least 120 minutes, at least 140 minutes, at least 160 minutes, at least 180 minutes, at least 200 minutes, at least 220 minutes, or about 240 minutes. In one embodiment, the pretreatment is conducted at a temperature(s) between about 120° C. and about 150° C. for a time between about 30 minutes and 240 minutes.

Using pretreatment temperatures between about 120° C. and about 150° C. for at least 60 minutes advantageously allows a significant amount of the lignin to become sulfonated. Using pretreatment temperatures between about 120° C. and about 150° C. for between 120 minutes and 240 minutes may promote significant hemicellulose dissolution and significant lignin dissolution, without producing excessive degradation products. The pretreatment time does not include the time to warm up the pretreatment liquor and the feedstock to at least 110° C.

In one embodiment, the aqueous pretreatment liquor is prepared by adding SO₂ to water, to an aqueous solution containing alkali, to an aqueous bisulfite salt solution, or to an aqueous slurry containing the softwood. In general, the SO₂ may be added as a gas, as an aqueous solution, or as a liquid (e.g., under pressure). When in an aqueous solution (e.g., dissolved in water), SO₂ may also be referred to as sulfurous acid (H₂SO₃). In one embodiment, the aqueous pretreatment liquor is prepared by adding commercially sourced SO₂, by adding SO₂ prepared on site (e.g., by burning sulfur), by adding recycled SO₂ (e.g., recovered from and/or reused within the process), by adding make-up SO₂ (e.g., used to top up the amount of SO₂ present), or any combination thereof. Optionally, the aqueous pretreatment liquor is prepared by adding one or more other acids (e.g., H₂SO₄, HCl, or lignosulfonic acid (LSA)) in addition to the SO₂.

In one embodiment, the aqueous pretreatment liquor is prepared by adding sufficient SO₂ to provide the aqueous pretreatment liquor with a pH of 1.3 or below (e.g., measured at ambient temperature). In one embodiment, the aqueous pretreatment liquor is prepared by adding sufficient SO₂ to provide the aqueous pretreatment liquor with a pH below about 1.3, below about 1.25, below about 1.2, below about 1.15, below about 1.1, below about 1.0, below about 0.9, or below about 0.8 (measured at ambient temperature). In one embodiment, the aqueous pretreatment liquor is prepared by adding sufficient SO₂ to provide the aqueous pretreatment liquor with a pH between about 1.3 and about 0.4 (measured at ambient temperature). In one embodiment, the aqueous pretreatment liquor is prepared by adding sufficient SO₂ to provide the aqueous pretreatment liquor with a pH between about 1.25 and about 0.7 (measured at ambient temperature).

In one embodiment, the pretreatment includes heating the softwood (e.g., wood chips, wood shavings, sawdust, and/or powder) at an elevated temperature in an aqueous pretreatment liquor containing SO₂, wherein the initial pH is about 1.3 or below about 1.3. The “initial pH” refers to the pH of the feedstock slurry, at ambient temperature, near the start of the pretreatment (e.g., after the SO₂ has been added). The initial pH may be substantially similar to the pH of the aqueous pretreatment liquor. In one embodiment, the pretreatment is conducted with an initial pH that is below about 1.3, below about 1.25, below about 1.2, below about 1.1, below about 1.0, below about 0.9, or below about 0.8. In one embodiment, the initial pH is between about 1.3 and about 0.4. In one embodiment, the initial pH is between about 1.25 and about 0.7.

In one embodiment, the aqueous pretreatment liquor is prepared by adding sufficient SO₂ to provide a SO₂ concentration above a certain level. In general, the SO₂ in the pretreatment liquor/slurry may be present as SO₂, H₂SO₃, HSO₃⁻, and/or SO₃²⁻, according to the following reactions:

SO₂+H₂O<=>H₂SO₃ (1)

H₂SO₃+H₂O<=>HSO₃⁻+H₃O⁺ (2)

HSO₃⁻+H₂O<=>SO₃²⁻+H₃O⁺ (3)

The “concentration of SO₂” or “SO₂ concentration”, takes into account contributions from SO₂, H₂SO₃, HSO₃⁻, and SO₃²⁻, expressed on a molar-equivalent-to-SO₂ basis, but expressed as weight percent SO₂. However, at the conditions used in the pretreatment (e.g., pH values less than about 1.3), the equilibrium in equation (3) will be shifted to the left and there will be negligible contributions from SO₃²⁻. The weight percent of SO₂ may be based on the weight of the pretreatment liquor (on liquor), or based on the weight of the dry feedstock (on dry solids). The pretreatment liquor weight includes the weight of moisture in the feedstock, but not the weight of the dry solids.

In one embodiment, the aqueous pretreatment liquor is prepared by adding sufficient SO₂ to provide a SO₂ concentration that is greater than about 6 wt % (on liquor), greater than about 6.5 wt % (on liquor), greater than about 7 wt % (on liquor), greater than about 7.5 wt % (on liquor), greater than about 8 wt % (on liquor), greater than about 8.5 wt % (on liquor), greater than about 9.0 wt % (on liquor), greater than 9.5 wt % (on liquor), greater than about 10 wt % (on liquor), greater than about 11 wt % (on liquor), greater than about 12 wt % (on liquor), greater than about 13 wt % (on liquor), or greater than about 13.5 wt % (on liquor). In one embodiment, sufficient SO₂ is added to provide a SO₂ concentration near the start of pretreatment that is between about 8.5 wt % and about 19.5 wt % (on liquor). In one embodiment, sufficient SO₂ is added to provide a SO₂ concentration near the start of pretreatment that is between about 9.4 wt % and about 19.5 wt % (on liquor).

In one embodiment, sufficient SO₂ is added to provide a SO₂ concentration near the start of pretreatment that is greater than about 60 wt % (on dry solids), greater than about 65 wt % (on dry solids), greater than about 70 wt % (on dry solids), greater than about 75 wt % (on dry solids), greater than about 80 wt % (on dry solids), greater than about 85 wt % (on dry solids), greater than about 90 wt % (on dry solids), greater than about 95 wt % (on dry solids), or greater than about 100 wt % (on dry solids).

The concentration of SO₂ based on dry solids may be determined using the consistency of the feedstock. In general, the term consistency refers to the amount of undissolved dry solids or “UDS” in a sample, and is often expressed as a ratio on a weight basis (wt:wt), or as a percent on a weight basis, for example, % (w/w), also denoted herein as wt %. For example, consistency may be determined by filtering and washing the sample to remove dissolved solids and then drying the sample at a temperature and for a period of time that is sufficient to remove water from the sample, but does not result in thermal degradation of the sample. The dry solids are weighed. The weight of water in the sample is the difference between the weight of the wet sample and the weight of the dry solids.

In one embodiment, the pretreatment is conducted at a solids consistency between about 5 wt % and about 40 wt %. In one embodiment, the pretreatment is conducted at a solids consistency between about 10 wt % and about 40 wt %. In one embodiment, the pretreatment is conducted at a solids consistency between about 20 wt % and about 40 wt %. In one embodiment, the pretreatment is conducted at a solids consistency between about 20 wt % and about 35 wt %. In one embodiment, the pretreatment is conducted at a solids consistency between about 10 wt % and about 25 wt %.

A SO₂ concentration that is between about 9.4 wt % and about 19.5 wt % (on liquor) corresponds to a SO₂ concentration that is between about 84.3 wt % and about 175.6 wt % (on dry solids) at a consistency of about 10 wt %, or between about 14.0 wt % and about 29.3 wt % (on dry solids) at a consistency of about 40 wt %, respectively. A consistency of about 10 wt % may correspond approximately to a liquid to solids ratio of about 9:1, whereas a consistency of about 20 wt % may correspond approximately to a liquid to solids ratio of about 4:1.

In general, the concentration of SO₂ (on liquor, or dry solids) may be determined using titration (e.g., with potassium iodate). However, as this may be challenging when relatively high SO₂ concentrations are achieved by introducing SO₂ into a pressurizable reactor, the concentration of SO₂ may be determined using the SO₂ loading. The “SO₂ loading” refers to the amount of SO₂ fed to the pretreatment per amount of dry lignocellulosic biomass fed to the system (e.g., as a weight percentage (wt %)). If the reactor has a large headspace (e.g., greater than 75% of the total reactor volume), the concentration of SO₂ can take into account the volume of the reactor headspace and partitioning of SO₂ into the vapour phase.

It has been found that lignin dissolution is improved when the pretreatment includes heating the softwood at an elevated temperature in an aqueous pretreatment liquor containing both SO₂ and bisulfite salt. Bisulfite salts, may for example, be formed by reacting an alkali (base) with aqueous SO₂, or by bubbling SO₂ into a solution containing alkali (base). For example, consider the following acid-base reaction:

H₂SO₃+MOH<=>MHSO₃+H₂O (4)

where M may be referred to as the counter cation. Some examples of alkali suitable for use providing the bisulfite salt include NaOH, NaHCO₃, Na₂CO₃, KOH, KHCO₃, K₂CO₃, CaCO₃, MgO, NH₃, etc.

In one embodiment, the aqueous pretreatment liquor is prepared by adding SO₂ and alkali. In general, the alkali may include any compound(s) that forms the desired bisulfite salt when SO₂ is present (e.g., NaHSO₃, KHSO₃, Ca(HSO₃)₂, Mg(HSO₃)₂, or (NH₄)HSO₃). In one embodiment, the alkali includes NaOH, NaHCO₃, Na₂CO₃, KOH, KHCO₃, K₂CO₃, CaCO₃, CaO, MgO, or NH₃. In one embodiment, the alkali is NaOH, CaO, MgO, or NH₄OH.

As the alkali may be provided as a hydroxide, carbonate salt, or other form, for comparative purposes, the “concentration of alkali” or “alkali concentration” may be expressed on a molar-equivalent-to-M basis, where M is the cation on a monovalent basis (Na⁺, K⁺, NH₄⁺, ½Ca²⁺, ½ Mg²⁺), but expressed as weight percent hydroxide (OH).

In one embodiment, sufficient alkali is added to provide an alkali concentration, near the start of pretreatment, that is at least about 0.05 wt %, at least about 0.1 wt %, at least about at least about 0.2 wt %, at least about 0.3 wt %, at least about 0.4 wt %, or at least about 0.5 wt %, each expressed as weight percent hydroxide on liquor (e.g., OH, on liquor). In one embodiment, sufficient alkali is added to provide an alkali concentration that is between about 0.01 wt % (OH, on liquor) and about 0.7 wt % (OH, on liquor). In one embodiment, sufficient alkali is added to provide an alkali concentration that is between about 0.05 wt % (OH, on liquor) and about 0.5 wt % (OH, on liquor). In one embodiment, sufficient alkali is added to provide an alkali concentration that is between about 0.1 wt % (OH, on liquor) and about 0.3 wt % (OH, on liquor). In one embodiment, sufficient alkali is added to provide an alkali concentration, near the start of pretreatment, that is between about 0 wt % and less than about 0.42 wt % (OH, on liquor).

The alkali concentration on liquor may be converted to the alkali on dry solids by taking the solids consistency into account. In one embodiment, sufficient alkali is added to provide an alkali concentration, near the start of pretreatment, that is at least about 0.10 wt %, at least about 0.5 wt %, at least about at least about 1 wt %, at least about 1.5 wt %, at least about 2 wt %, at least about 2.5 wt %, at least about 3 wt %, at least about 3.5 wt %, at least about 4 wt %, at least about 5 wt %, or at least about 6 wt %, each expressed as weight percent hydroxide on dry solids (e.g., OH, on dry solids). In one embodiment, sufficient alkali is added to provide an alkali concentration, near the start of pretreatment, that is between about 0.50 wt % and about 3 wt % (OH, on dry solids).

For reference, if alkali is provided only by adding NaOH, an alkali concentration of about 0.16 wt % (OH, on liquor) may be roughly equivalent to a NaOH charge of about 0.38 wt % (on liquor) or a NaHSO₃ charge of about 1 wt % (on liquor). A NaHSO₃ charge of about 1% (on liquor) corresponds to a NaHSO₃ charge of about 9 wt % (on dry solids) when the consistency is about 10 wt %, about 4 wt % (on dry solids) when the consistency is about 20 wt %, or about 1.5 wt % (on dry solids) when the consistency is about 40 wt %.

In general, the alkali concentration in the aqueous pretreatment liquor may include contributions from alkali inherent to the feedstock (e.g., K₂CO₃, CaCO₃, and/or Na₂CO₃) and/or alkali added for the pretreatment (e.g., NaOH, CaO, MgO, NH₃, etc.). For example, without adding alkali and without washing, wheat straw may have an inherent alkali concentration that is between about 0.15 wt % and about 0.63 wt % (OH, on dry solids), whereas bagasse may provide an inherent alkali concentration as high as about 0.2 wt % (OH, on dry solids). However, since woody feedstock tends to have a much lower inherent alkali concentration, the inherent alkali in softwood feedstock may be negligible.

The pH of the pretreatment liquor and/or the pH of the feedstock slurry near the start of pretreatment may be dependent on the amount of SO₂ (and/or other acids) and/or the amount of alkali present. In one embodiment, the pretreatment liquor is prepared by adding alkali to water or to an aqueous solution of SO₂ such that ratio of SO₂ to alkali results in excess SO₂ (e.g., such that the pH is below about 1.3, below about 1.2, below about 1.1, or below about 1.0). In one embodiment, the pH (e.g., of pretreatment liquor and/or initial) is achieved by selecting an appropriate ratio of SO₂ to alkali. In one embodiment, the ratio of the concentration of SO₂ to the concentration of alkali (both mass on dry solids, or mass on liquor, where the concentration of alkali is expressed as weight percent hydroxide) is greater than 30, greater than 35, greater than 40, greater than 45, or greater than 50.

Pretreating with SO₂ and bisulfite salt is advantageous because it may promote sulfonation of the lignin, thereby modifying the structure of the lignin, and/or may promote lignin and/or hemicellulose dissolution. In sulfonating lignin, lignosulfonic acid may be produced. Lignosulfonic acid is a strong acid that may promote hemicellulose dissolution. Since lignosulfonic acid is a stronger acid than SO₂, the pH of the slurry may drop as the pretreatment progresses (e.g., from some initial pH to some final pH).

In one embodiment, the amount of SO₂ and alkali added provides a slurry of pretreated material (pretreated slurry) having a pH less than about 1 (e.g., final pH is less than about 1). In one embodiment, the amount of SO₂ and alkali added provides a pretreated slurry having a pH less than about 0.9, less than about 0.8, less than about 0.7, less than about 0.6, or less than about 0.5. In one embodiment, the amount of SO₂ and alkali added provides a pretreated slurry having a pH between about 1 and about 0.3. The “final pH” refers to the pH of the pretreated slurry, at ambient temperature, at the end of the pretreatment (e.g., after the pretreated material is discharged from the pretreatment reactor(s)).

Although low pH values have been previously associated with excessive acid-catalyzed hydrolysis of hemicellulose and/or cellulose, and/or with the formation of an excessive amount of potential fermentation inhibitors (e.g., furfural and hydroxymethylfurfural (HMF)), it has been found that good glucose yields and reasonable xylose yields may be achieved when subjecting bagasse or wheat straw to a relatively low temperature SO₂ pretreatment (e.g., below 160° C.) at low pH (e.g., below 1.3) when the SO₂ loading is relatively high. Advantageously, these good results can be obtained without having to add an organic solvent (e.g., ethanol).

However, given the reputation of softwood, and in particular resinous softwood, for being unsuitable for acid sulfite pulping at low pH values (e.g., below 1.4), it is surprising that heating softwood (e.g., wood chips, wood shavings, sawdust, and/or powder) at an elevated temperature in an aqueous pretreatment liquor containing both SO₂ and bisulfite salt, where the pH is less than 1.3, could significantly improve hydrolysis.

Without being bound by theory, the improvement in hydrolysis may be related to the relative high SO₂ concentration (near the start of pretreatment). For example, it has now been found that providing a concentration of SO₂ greater than about 75 wt % (on dry solids), or greater than about 8.4 wt % (on liquor) (e.g., when the consistency of slurry is about 10 wt %, and when the liquor has a NaHSO₃ concentration of 10 g/L), can provide a good pretreatment for pine.

In general, the SO₂, alkali, bisulfite salt, water, and/or feedstock may be added in any order, or simultaneously, to the pretreatment reactor. For example, the aqueous pretreatment liquor may be prepared prior to being introduced to the pretreatment reactor, within the pretreatment reactor, or a combination thereof. In one embodiment, the aqueous pretreatment liquor containing SO₂, alkali, and water is prepared in one or more vessels prior to being introduced into the pretreatment reactor (e.g., which may or may not already contain the feedstock).

Preparing an aqueous pretreatment liquor containing SO₂ and alkali prior to introducing it into the pretreatment reactor may facilitate providing higher SO₂ concentrations and/or pre-warming of the pretreatment liquor. In general, the concentration of a SO₂ solution may be limited by the solubility of SO₂ in water. For example, if no alkali is added, the SO₂ concentration may be limited to below about 10 wt % (on liquor) at about 23° C. The SO₂ concentration may be increased by cooling the water or aqueous alkali solution prior to bubbling in SO₂. Alternatively, or additionally, a higher SO₂ concentration may be obtained by introducing the SO₂ under pressure. In one embodiment, SO₂ is introduced into a vessel to provide an SO₂ partial pressure of about 18 psia to about 37 psia, at 25° C. In any case, the pretreatment liquor may or may not be heated prior to entering the pretreatment reactor (e.g., heated under pressure).

In one embodiment, the aqueous pretreatment liquor is prepared using one or more vessels prior to being introduced into the pretreatment reactor. For example, in one embodiment, the aqueous pretreatment liquor is prepared using one or more tanks. In one embodiment, the aqueous pretreatment liquor is prepared using an accumulator, surge tank, and/or buffer tank. Accumulators (or surge tanks), may for example, be used to collect relief gases (e.g., rich in SO₂) for direct reuse. Such relief gases may result when it is necessary to vent the pretreatment reactor as the temperature rises.

In one embodiment, the aqueous pretreatment liquor is prepared by feeding SO₂ into water or an aqueous solution containing alkali contained in some vessel (e.g., absorption tower). In one embodiment, SO₂ is bubbled into a cooled alkali solution. In one embodiment, this SO₂/alkali solution is transferred to a pressure accumulator where heat, steam, and/or additional SO₂ (e.g., from a relief valve) are added. In one embodiment, the heated pretreatment liquor from the accumulator is introduced into the pretreatment reactor containing the softwood feedstock (e.g., woodchips). In one embodiment, the softwood is pre-steamed prior to adding the heated pretreatment liquor. In one embodiment, the softwood is not pre-steamed prior to adding the heated pretreatment liquor. In one embodiment, the heated pretreatment liquor and softwood feedstock are heated (e.g., to a temperature between about 110° C. and about 160° C.) in the pretreatment reactor.

In general, the pretreatment may be carried out in batch mode, semi-batch mode, or continuous mode, in one or more pretreatment reactors. The pretreatment reactor(s) may be of any suitable construction. For example, the pretreatment may be conducted in one or more vertical reactors, horizontal reactors, inclined reactors, or any combination thereof. In one embodiment, the pretreatment is carried out in batch mode in a steam autoclave. In one embodiment, the pretreatment is conducted in continuous mode in a plug flow reactor. In one embodiment, the pretreatment is conducted in a continuous mode horizontal screw fed reactor. In one embodiment, the pretreatment is conducted in a counter-current flow reactor. In one embodiment, the pretreatment is conducted in a digester (e.g., batch or continuous). Such digester may be of any suitable conventional construction (e.g., used in wood pulping).

In one embodiment, the pretreatment is conducted in a pretreatment system and/or reactor that includes a heater, or some other heating means, for heating the feedstock. Such heating may be direct or indirect (e.g., direct steam heating or indirect steam heating). In one embodiment, the pretreatment reactor and/or the pretreatment system includes direct steam injection inlets (e.g., from a low pressure boiler). For example, in one embodiment, the pretreatment reactor is a digester that provides direct steam injection at the bottom of the digester, with heat transfer throughout the rest of the digester occurring by convection. In one embodiment, the pretreatment reactor is heated by indirect steam heating via the use of one or more heat-exchangers and forced liquor circulation (e.g., using liquid circulation loops). For example, in one embodiment, the aqueous pretreatment liquor is removed from the digester through a screen, and returned to the digester via a pipe, after the circulating liquid is heated with a heat exchanger couple to the pipe.

In one embodiment, the pretreatment is conducted in a pretreatment reactor and/or system that is pressurizable (e.g., a digester). For example, in one embodiment, the pretreatment reactor and/or pretreatment system includes a plurality of valves and/or other pressure increasing, pressure decreasing, or pressure maintaining components for providing and/or maintaining the pretreatment reactor at a specific pressure. Conventional digesters used in wood pulping are generally pressurizable.

In one embodiment, the pretreatment includes adding steam to provide a total pressure between about 190 psia and about 630 psia, between about 200 psia and about 600 psia, between about 250 psia and about 550 psia, or between about 300 psia and about 500 psia. For example, in one embodiment, where the total pressure is about 190 psia, the partial pressure of SO₂ may be about 21 psia, whereas the steam partial pressure may be about 169 psia.

In one embodiment, the pretreatment is conducted in a pretreatment reactor and/or system that includes a batch digester. In batch cooking, woodchips and pretreatment liquor may be added to the digester and the contents heated at some pretreatment temperature for some pretreatment time. Batch digesters may be heated by indirect and/or direct steam heating. Following the pretreatment, the pretreated material may be blown from the bottom of the digester (e.g., which may be conical in shape to improve discharge). In one embodiment, the batch digester is a single, cylindrically shaped vessel. In one embodiment, the batch digester has a diameter between 2.5 and 5 meters, a height between 8.5 and 19 meters, and a volume between 70 and 400 m³.

In one embodiment, the pretreatment is conducted in a pretreatment reactor and/or system that includes a continuous digester. In continuous cooking, the woodchips and pretreatment liquor may be fed at a rate that allows the pretreatment reaction to be complete by the time the materials exit the reactor. Continuous digesters may be single vessels or multi-vessel systems. For example, a single vessel may have an impregnation zone, one or more cooking zones, and a wash zone. In the impregnation zone the pretreatment liquor may penetrate and diffuse into the woodchips. In the cooking zone, the woodchips and pretreatment liquor may flow in co-current or counter-current directions. In the wash zone, cooler spent liquor may be used to displace hot spent liquor. In a multi-vessel system, the impregnation zone may be a separate vessel.

In one embodiment, the pretreatment is conducted in a pretreatment reactor (e.g., digester) having a basket for holding the woodchips. In one embodiment, the feedstock is placed in the basket and is pre-steamed (e.g., for 60-90 mins). Pre-steaming the feedstock may drive out air and/or may pre-warm the feedstock (e.g., to about 90° C.). In one embodiment, the pre-steamed feedstock is drained (e.g., to remove the condensate) prior to introducing the aqueous pretreatment liquor. In one embodiment, pre-prepared pretreatment liquor (e.g., at or below ambient temperature) is added to the feedstock in the desired liquor to wood ratio (e.g., 9:1, 8:1, 7:1, 6:1, 5:1, 4:1, 3:1, 2:1, etc.), and the resulting slurry heated to the pretreatment temperature. In one embodiment, pre-warmed pretreatment liquor is added to the feedstock (e.g., which is optionally pre-warmed and/or pre-steamed) in the desired liquor to wood ratio (e.g., 9:1, 8:1, 7:1, 6:1, 5:1, 4:1, 3:1, 2:1, etc.), and the resulting slurry heated to the pretreatment temperature.

In one embodiment, the pretreated material is discharged from the pretreatment reactor under pressure (e.g., blow down). In one embodiment, the discharged pretreated material is collected in a receiving vessel (e.g., a flash tank or blow tank, which may or may not be at atmospheric pressure). In one embodiment, the discharged pretreated material is collected in a diffusion washer. In one embodiment, the discharged pretreated material is fed for downstream processing.

### Preparing the Pretreated Material for Enzymatic Hydrolysis

In one embodiment, the pretreated material is subject to one or more optional steps to prepare it for enzymatic hydrolysis. For example, in one embodiment the pretreated material is subject to a pressure reduction (e.g., flashing), a liquid/solid separation (e.g., filtering), a washing step, a cooling step, mechanical refining, and/or a pH adjustment step.

In one embodiment, the pretreated biomass is subject to a pressure reduction. For example, in one embodiment, the pressure is reduced using one or more flash tanks in fluid connection with the pretreatment reactor. Flashing may reduce the temperature of the pretreated biomass to about 100° C. if an atmospheric flash tank, or lower if a vacuum flash tank.

In one embodiment, the pretreated biomass is subject to a solid/liquid separation, which provides a solid fraction and a liquid fraction. The solid fraction may contain undissolved solids such as unconverted cellulose and/or insoluble lignin. The liquid fraction, which may also be referred to as the pretreatment hydrolysate, may contain soluble compounds such as sugars (e.g., mannose, xylose, glucose, and arabinose), organic acids (e.g., acetic acid and glucuronic acid), soluble lignin (e.g., lignosulfonates), soluble sugar degradation products (e.g., furfural, which may be derived from C5 sugars, and HMF, which may be derived from C6 sugars), salts (e.g., sulfite salts), and/or small amounts of wood extractives. Exemplary solid/liquid separation methods include, but are not limited to, filtration, membrane filtration, tangential flow filtration (TFF), centrifugation, sedimentation, and flotation. For example, in one embodiment, the pretreated material fed to one or more centrifuges that provide a solid stream and a liquid stream. In one embodiment, the solid/liquid separation uses vacuum or pressure to facilitate the separation. For example, in one embodiment, the pretreated material fed to a filter press or belt press. In one embodiment, the solid/liquid separation is conducted in batch, continuous, or dis-continuous mode.

In one embodiment, the pretreated material is subject to one or more washing steps. In one embodiment, the solid fraction from a solid/liquid separation is washed to remove soluble components, including potential inhibitors and/or inactivators. Washing may also remove soluble lignin (e.g., sulfonated lignin). In one embodiment, the pretreated material is washed as part of the liquid/solid separation (e.g., as part of decanter/wash cycle). The pretreated material may be washed as part of the liquid/solid separation at high or low pressure, which may or may not reduce the temperature of the pretreated material. In one embodiment, the wash water is reused or recycled. In one embodiment, the wash water is combined with the liquid fraction and sent for further processing.

In one embodiment, the pretreated material is subjected to one or more cooling steps. For example, in one embodiment, the pretreated material (e.g., liquid fraction, solid fraction, or whole slurry) is cooled to within a temperature range compatible with enzyme(s) added for the enzymatic hydrolysis. For example, conventional cellulases often have an optimum temperature range between about 40° C. and about 65° C., and more commonly between about 50° C. and 65° C., whereas thermostable and/thermophilic enzymes may have optimum temperatures that are much higher (e.g., as high as, or greater than 80° C.). In one embodiment, the pretreated biomass is cooled to within a temperature range compatible with enzyme(s) and yeast used in a simultaneous saccharification and fermentation (SSF).

In general, the one or more cooling steps may include passive and/or active cooling of the liquid fraction, the solid fraction, or a combination of the liquid and solid fraction. In one embodiment, the one or more cooling steps include flashing, heat exchange, washing, etc. In one embodiment, cooling is provided by injecting a fluid into the pretreated biomass. For example, in one embodiment, cooling is achieved when alkali and/or water is added to the pretreated biomass into order to provide the pH and/or consistency desired for enzymatic hydrolysis. Advantageously, since the pretreatment is conducted at relatively low temperatures (e.g., between 110° C. and 160° C.), the one or more cooling steps may not have to produce a significant temperature drop.

In one embodiment, the pretreated material is subjected to one or more mechanical refining steps. For example, in one embodiment, the pretreated material (e.g., solid fraction or whole slurry) is subject to a mechanical size reduction using disk refining, which may for example, fiberize the pretreated woodchips for the following enzymatic hydrolysis. Disk refining, may for example, be advantageous for large chips. In one embodiment, disk refining is conducted on the solid fraction after the solid/liquid separation and/or washing.

In one embodiment, the pretreated material is subjected to one or more pH adjustment steps. In one embodiment, the pH of the pretreated biomass is adjusted to within a range near the pH optimum of the enzyme(s) used in hydrolysis. For example, cellulases typically have an optimum pH range between about 4 and about 7, more commonly between about 4.5 and about 5.5, and often about 5. In one embodiment, the pH is adjusted to between about 4 and about 8. In one embodiment, the pH is adjusted to between about 4.5 and about 6. In one embodiment, the pH is adjusted so as to substantially neutralize the pretreated biomass.

In one embodiment, the pH of the pretreated biomass is increased as a result of the washing step. In one embodiment, the pH of the pretreated biomass is increased by adding alkali (e.g., calcium hydroxide, potassium hydroxide, sodium hydroxide, ammonia gas, etc.). For example, in one embodiment, sufficient alkali is added to increase the pH of the pretreated biomass to a pH near the optimum pH range of the enzyme(s) used in hydrolysis. In one embodiment, the pH adjustment step includes adding sufficient alkali to overshoot the optimum pH of the enzyme (e.g., overliming), and then adding acid to reduce the pH to near the optimum pH range of the enzyme(s). In one embodiment, the pH adjustment includes flashing and/or a heat treatment to drive SO₂ out of solution.

In general, the pH adjustment step may be conducted on the solid fraction, the liquid fraction, and/or a combination thereof, and may be conducted before, after, and/or as part of the one or more cooling steps. For example, in embodiments wherein the pretreated material is separated into a solid fraction and a liquid fraction, where only the solid fraction is fed to enzymatic hydrolysis, the pH of the liquid fraction may require adjustment prior to being fed to fermentation (e.g., separate from, or with, the hydrolysate from the solid fraction). For example, in one embodiment, the pH of the liquid fraction is adjusted to the pH optimum of the microorganism used in a subsequent fermentation step. For example, Saccharomyces cerevisiae may have optimum pH values between about 4 and about 5.5.

In general, the pretreated material prepared for and fed to enzymatic hydrolysis may include the solid fraction, the liquid fraction, or some combination thereof. For example, although the primary goal of enzymatic hydrolysis is to convert the cellulose in the solid fraction to glucose, it may be advantageous to also include the liquid fraction. For example, by feeding the whole pretreated slurry (e.g., cooled and pH adjusted) to enzymatic hydrolysis the solid/liquid separation step can be avoided. Moreover, a washing step can be avoided. While washing may remove potential inhibitors and/or inactivators, and thus may increase enzyme efficiency, it may also remove fermentable sugars, and thus reduce ethanol yield. Providing little or no washing of the pretreated biomass is advantageous in that it requires less process water and provides a simpler process.

### Enzymatic Hydrolysis

In one embodiment, the pretreated material is fed to one or more enzymatic hydrolysis reactors, wherein cellulose in the solid fraction is converted to glucose. In one embodiment, the pretreated material fed to one or more enzymatic hydrolysis reactors includes washed solids (e.g., washed with water in order to remove most of the pretreatment hydrolyzate). In one embodiment, the pretreated material fed to one or more enzymatic hydrolysis reactors includes the whole slurry (e.g., where the liquid and solid fractions were not separated). In this embodiment, the whole slurry of pretreated material may be pH adjusted, detoxified, and/or diluted. In one embodiment, the pretreated slurry is filtered, and the solids are partially and/or minimally washed.

In one embodiment, enzyme is added to and/or mixed with the pretreated material prior to entering the enzymatic hydrolysis reactor and/or within the enzymatic hydrolysis reactor. In one embodiment, enzyme addition is achieved by adding enzyme to a reservoir, such as a tank, to form an enzyme solution, which is then introduced to the pretreated material. In one embodiment, enzyme addition is after cooling and alkali addition. In one embodiment, enzyme addition includes the addition of cellulase.

Cellulases are enzymes that can break cellulose chains into glucose. The term “cellulase”, as used herein, includes mixtures or complexes of enzymes that act serially or synergistically to decompose cellulosic material, each of which may be produced by fungi, bacteria, or protozoans. For example, in one embodiment, the cellulase is an enzyme cocktail comprising exo-cellobiohydrolases (CBH), endoglucanases (EG), and/or β-glucosidases (PG), which can be produced by a number of plants and microorganisms. In one embodiment, the cellulase is a commercial cellulase obtained from fungi of the genera Aspergillus, Humicola, Chrysosporium, Melanocarpus, Myceliopthora, Sporotrichum or Trichoderma, or from bacteria of the genera Bacillus or Thermobifida. In addition to CBH, EG and βG, the cellulase may include several accessory enzymes that may aid in the enzymatic digestion of cellulose, including glycoside hydrolase 61 (GH61), swollenin, expansin, lucinen, and cellulose-induced protein (Cip). In one embodiment, the enzyme includes a lytic polysaccharide monooxygenase (LPMO) enzyme. For example, in one embodiment, the enzyme includes GH61. In one embodiment, the cellulase is a commercial cellulase composition that is suitable for use in the methods/processes described herein. In one embodiment, one or more cofactors are added. In one embodiment, O₂ or H₂O₂ is added. In one embodiment, ascorbic acid or some other reducing agent is added. In one embodiment, the pH is adjusted during the enzymatic hydrolysis.

In general, the enzyme dose may depend on the activity of the enzyme at the selected pH and temperature, the reaction time, and/or other parameters. It should be appreciated that these parameters may be adjusted as desired by one of skill in the art. In one embodiment, cellulase is added at a dosage between about 1 to 20 mg protein per gram cellulose (mg/g), at a dosage between about 2 to 20 mg protein per gram cellulose, at a dosage between about 1 to 15 mg protein per gram cellulose, or at a dosage between about 1 to 10 mg protein per gram cellulose. The protein may be quantified using either the bicinchoninic acid (BCA) assay or the Bradford assay.

In one embodiment, the initial concentration of cellulose in the slurry, prior to the start of enzymatic hydrolysis, is between about 0.01% (w/w) to about 20% (w/w). In one embodiment, the slurry fed to enzymatic hydrolysis is at a solids content between about 10% and 25%.

In one embodiment, the enzymatic hydrolysis is carried out at a pH and temperature that is at or near the optimum for the added enzyme. In one embodiment, the enzymatic hydrolysis is carried out at one or more temperatures between about 30° C. and about 95° C., between about 45° C. and about 65° C., between about 45° C. and about 55° C., or between about 50° C. and about 65° C. In one embodiment, the enzymatic hydrolysis is carried such that the pH value during the hydrolysis is between about 3.5 and about 8.0, between about 4 and about 6, or between about 4.8 and about 5.5. In one embodiment, the enzymatic hydrolysis is carried out for a time between about 10 and about 250 hours, or between about 50 and about 250 hours. In one embodiment, the enzymatic hydrolysis is carried out for at least 24 hours, at least 36 hours, at least 48 hours, at least 72 hours, or at least 80 hours. In general, conducting the enzymatic hydrolysis for at least 24 hours may promote hydrolysis of both the amorphous and crystalline cellulose.

In one embodiment, the enzymatic hydrolysis includes agitation. Agitation may improve mass and/or heat transfer and may provide a more homogeneous enzyme distribution. In addition, agitation may entrain air in the slurry, which may be advantageous when the enzyme contains a LPMO. In one embodiment, air and/or oxygen is added to the hydrolysis. In one embodiment, air and/or oxygen is added to the hydrolysis using a pump or compressor in order to maintain the dissolved oxygen concentration within a range that is sufficient for the full activity of LPMOs or any other oxygen-dependent components of the catalyst used to effect hydrolysis. In one embodiment, air or oxygen is bubbled into the slurry or headspace of one or more of the hydrolysis reactors.

In one embodiment, the enzymatic hydrolysis is conducted as a batch process, a continuous process, or a combination thereof. In one embodiment, the enzymatic hydrolysis is agitated, unmixed, or a combination thereof. In one embodiment, the enzymatic hydrolysis is conducted in one or more dedicated hydrolysis reactors, connected in series or parallel. In one embodiment, the one or more hydrolysis reactors are jacketed with steam, hot water, or other heat sources.

In one embodiment, the enzymatic hydrolysis is conducted in one or more continuous stirred tank reactors (CSTRs) and/or one or more plug flow reactors (PFRs). In plug flow reactors, the slurry is pumped through a pipe or tube such that it exhibits a relatively uniform velocity profile across the diameter of the pipe/tube and such that residence time within the reactor provides the desired conversion. In one embodiment, the hydrolysis includes a plurality of hydrolysis rectors including a PFR and a CSTR in series.

In one embodiment, the enzymatic hydrolysis and fermentation are conducted in separate vessels so that each biological reaction can occur at its respective optimal temperature. In one embodiment, the enzymatic hydrolysis and fermentation are conducted is a same vessel, or series of vessels.

In one embodiment, the hydrolysate provided by enzymatic hydrolysis is filtered to remove insoluble lignin and/or undigested cellulose.

### Fermentation

In one embodiment, the glucose produced during enzymatic hydrolysis is fermented via one or more microorganisms. In one embodiment, the mannose and/or other sugars produced during pretreatment is fermented via one or more microorganisms. In one embodiment, the glucose produced during enzymatic hydrolysis is fermented together with, or separately, from the sugars produced during pretreatment. For example, in one embodiment, where the whole slurry is fed to enzymatic hydrolysis, the hydrolysate is subject to a fermentation such that the glucose produced from the cellulose and the mannose produced from the hemicellulose are fermented together. In one embodiment, the fermentation microorganism(s) includes include any suitable yeast and/or bacteria.

In one embodiment, at least a portion of the hydrolysate produced during enzymatic hydrolysis is subjected to a fermentation with Saccharomyces spp. yeast. For example, in one embodiment, the fermentation is carried out with Saccharomyces cerevisiae, which has the ability to utilize a wide range of sugars such as glucose, fructose, mannose, sucrose, galactose, maltose, and maltotriose to produce a high yield of ethanol. In one embodiment, the glucose and/or other hexoses derived from the cellulose are fermented to ethanol using a wild-type Saccharomyces cerevisiae or a genetically modified yeast. In one embodiment, the fermentation is carried out with Zymomonas mobilis bacteria.

In one embodiment, at least a portion of the hydrolysate produced during enzymatic hydrolysis is fermented by one or more microorganisms to produce a fermentation broth containing butanol. For example, in one embodiment the glucose produced during enzymatic hydrolysis is fermented to butanol with Clostridium acetobutylicum.

In one embodiment, one or more of the sugars produced during the pretreatment (e.g., in the pretreatment hydrolysate) is fermented to ethanol using one or more microrganisms. For example, in one embodiment, xylose and/or arabinose produced during the pretreatment is fermented to ethanol with a yeast strain that naturally contains, or has been engineered to contain, the ability to ferment these sugars to ethanol. Examples of microbes that have been genetically modified to ferment xylose include recombinant Saccharomyces strains into which has been inserted either (a) the xylose reductase (XR) and xylitol dehydrogenase (XDH) genes from Pichia sapites.

In one embodiment, the xylose and other pentose sugars produced during the pretreatment are fermented to xylitol by yeast strains selected from the group consisting of Candida, Pichia, Pachysolen, Hansenula, Debaryomyces, Kluyveromyces and Saccharomyces.

In general, the hydrolysate from the enzymatic hydrolysis and the pretreatment hydrolysate can be subjected to separate fermentations or a combined fermentation. For example, consider the case where the pretreated biomass is subject to a solid/liquid separation and only the solid fraction is fed to enzymatic hydrolysis. In this case, the glucose produced by enzymatic hydrolysis can be fermented on its own, or can be combined with the liquid fraction and then fermented.

For example, for softwood, the enzymatic hydrolysate may contain primarily glucose, whereas the pretreatment hydrolysate may contain primarily mannose, both which may be fermented to ethanol using Saccharomyces cerevisiae. In one embodiment, the hydrolysate from the enzymatic hydrolysis and the pretreatment hydrolysate are combined and fed to a fermentation using Saccharomyces cerevisiae.

The pretreatment hydrolysate from softwood, which includes solubilized hemicellulose, may also contain C5 sugars such as xylose. In one embodiment, the hydrolysate from the enzymatic hydrolysis and the pretreatment hydrolysate are combined and fed to a fermentation using C5 utilizing and ethanol producing yeasts (e.g., such as Pichia fermentans and Pichia stipitis) that are cocultured with Saccharomyces cerevisiae. In one embodiment, the hydrolysate from the enzymatic hydrolysis and the pretreatment hydrolysate are combined and fed to a fermentation using genetically engineered Saccharomyces yeast capable of cofermenting glucose and xylose.

In general, the dose of the microorganism(s) will depend on a number of factors, including the activity of the microorganism, the desired reaction time, and/or other parameters. It should be appreciated that these parameters may be adjusted as desired by one of skill in the art to achieve optimal conditions. In one embodiment, the fermentation is supplemented with additional nutrients required for the growth of the fermentation microorganism. For example, yeast extract, specific amino acids, phosphate, nitrogen sources, salts, trace elements and vitamins may be added to the hydrolysate slurry to support their growth. In one embodiment, yeast recycle is employed.

In one embodiment, the fermentation is carried out at a pH and temperature that is at or near the optimum for the added microorganism. For example, Saccharomyces cerevisiae may have optimum pH values between about 4 and about 5.5 and a temperature optimum between about 25° C. and about 35° C. In one embodiment, the fermentation is carried out at one or more temperatures between about 25° C. to about 55° C. In one embodiment, the fermentation is carried out at one or more temperatures between about 30° C. to about 35° C.

In general, the fermentation may be conducted as a batch process, a continuous process, or a fed-batch mode. For example, in one embodiment, the fermentation is conducted in continuous mode, which may offer greater productivity and lower costs. In one embodiment, the enzymatic hydrolysis may be conducted in one or more fermentation tanks, which can be connected in series or parallel. In general, the fermentation may be agitated, unmixed, or a combination thereof. For example, in one embodiment, the fermentation is conducted one or more continuous stirred tank reactors (CSTRs) and/or one or more plug flow reactors (PFRs). In one embodiment, the one or more fermentation tanks are jacketed with steam, hot water, or other heat sources.

In one embodiment, the enzymatic hydrolysis and fermentation are conducted in separate vessels so that each biological reaction can occur at its respective optimal temperature. In another embodiment, the hydrolysis (e.g., which may be also referred to as saccharification) is conducted simultaneously with the fermentation in same vessel. For example, in one embodiment, a simultaneous saccharification and fermentation (SSF) is conducted at temperature between about 35° C. and 38° C., which is a compromise between the 50° C. to 55° C. optimum for cellulase and the 25° C. to 35° C. optimum for yeast.

### Fermentation Product Recovery

In one embodiment, the fermentation product is recovered. For example, in one embodiment, the fermentation product is an alcohol and is subject to an alcohol recovery process wherein the alcohol is concentrated and/or purified from the fermented solution. In one embodiment, the fermentation broth is subject to a solid/liquid separation (e.g., filtered) and the liquid fraction is fed to a distillation system. In one embodiment, the fermentation broth, which may include unconverted cellulose, insoluble lignin, and/or other undissolved substances, is fed to the distillation system without being pre-filtered.

In one embodiment, the fermentation produces ethanol, which is recovered using one or more distillation columns that separate the ethanol from other components (e.g., water). In general, the distillation column(s) in the distillation unit may be operated in continuous or batch mode, although are typically operated in a continuous mode. Heat for the distillation process may be introduced at one or more points, either by direct steam injection or indirectly via heat exchangers. After distillation, the water remaining in the concentrated ethanol stream (i.e., vapour) may be removed from the ethanol rich vapour by a molecular sieve resin, by membrane extraction, or other methods known to those of skill in the art for concentration of ethanol beyond the 95% that is typically achieved by distillation (e.g., a vapour phase drying). The vapour may then be condensed and denatured.

### Sulfur Dioxide Recovery

Excess SO₂ not consumed during the pretreatment can be recovered and/or recycled. For example, in one embodiment, SO₂ not consumed during the pretreatment is forced out of the pretreated slurry by a pressure reduction (e.g., top relief, atmospheric flash, vacuum flash, vacuum, etc.) or by a temperature increase (e.g., evaporation by heating). The SO₂ forced out of the pretreated slurry can be collected, recovered, and/or recycled within the process. In one embodiment, the SO₂ forced out of the pretreated slurry is fed to an SO₂ recovery unit. For example, in one embodiment, the slurry of pretreated material is flashed, and the flash stream, which contains the excess SO₂, is fed to a SO₂ recovery unit. In one embodiment, the SO₂ forced out of the pretreated slurry is reused directly (e.g., fed to an accumulator or the pretreatment reactor).

In general, the SO₂ recovery unit may be based on any suitable SO₂ recovery technology, as known in the art. In one embodiment, the SO₂ recovery unit includes a partial condenser, an SO₂ stripper, and/or an SO₂ scrubbing system. In one embodiment, the SO₂ recovery unit includes a SO₂ scrubbing system, which may include one or more packed absorbers (e.g., amine-based, alkali-based, or other absorbers). In one embodiment, the SO₂ recovery unit provides purified SO₂ that can be recycled for use in the pretreatment. In one embodiment, the SO₂ recovery unit provides partially purified SO₂ that can be recycled for use in the pretreatment.

In one embodiment, the recovered SO₂, which is optionally stored temporarily, is recycled directly back into the process. Advantageously, SO₂ recovery allows the recycling of sulfur within the system, and thus improves the process economics (e.g., since less SO₂ needs to be acquired for pretreatment).

### Additional Product Recovery

Although a key goal of the process is to convert cellulose to glucose, which may then be converted to a fermentation product, one or more other products may be produced during the process. Softwood may, for example, contain about 40-45% cellulose, about 27% hemicellulose, and about 27% lignin. Producing one or more additional products, and/or improving the yield of glucose/fermentation product, from the non-cellulose components may be advantageous.

Depending on the pretreatment conditions, in addition to unconverted cellulose, the pretreated slurry may contain hemicellulose sugars (e.g., mannose, xylose, glucose), organic acids (e.g., acetic acid), soluble lignin (e.g., lignosulfonate), soluble sugar degradation products (e.g., furfural and HMF), and/or one or more salts (e.g., sulfite salts).

In one embodiment, one or more products derived from the hemicellulose sugars are produced and/or recovered. For example, in one embodiment, wherein the pretreated slurry is subject to a solid/liquid separation and the solids are fed to enzymatic hydrolysis, the liquid fraction may be subject to separate processing.

In one embodiment, the liquid fraction is pH adjusted, detoxified, and/or cooled prior to being fed to a fermenter. In this embodiment, the hemicellulose sugars are fermented separately from the glucose produced during enzymatic hydrolysis. Advantageously, this embodiment may improve the fermentation product (e.g., ethanol) yield.

In one embodiment, the liquid fraction is fed to an anaerobic digester, wherein the organic contents may be converted to biogas. In one embodiment, the liquid fraction is fed to a wet oxidation, wherein the organic contents may be converted to acetic acid or acetate. In one embodiment, the biogas and/or acetic acid is used as a feedstock to produce ethanol via a gas fermentation that uses carbon monoxide, carbon dioxide, and/or hydrogen as a substrate. Advantageously, this improves the ethanol yield as ethanol is produced from the cellulose component in addition to the hemicellulose and/or lignin components. In one embodiment, the biogas is used within the process in order to reduce greenhouse gas emissions. In one embodiment, the biogas is upgraded to pipeline standards and provided or allocated for transportation use or for use in producing a transportation fuel. This embodiment is particularly advantageous because in using a pretreatment liquor having a pH below about 1.3 and a relatively high SO₂ concentration, both the hemicellulose and lignin dissolution are improved, which may improve the product yield from these fractions.

In one embodiment, lignosulfonate generated during the pretreatment is recovered. The term lignosulfonate refers to water soluble sulfonated lignin (i.e., soluble in water at neutral and/or acid conditions) and encompasses both lignosulfonic acid and its neutral salts. In general, lignosulfonate may be recovered following pretreatment, enzymatic hydrolysis, and/or fermentation. In one embodiment, lignosulfonate is recovered for energy production (e.g., combusted). In one embodiment, lignosulfonate is recovered for producing value-added materials (e.g., a dispersing agent, a binding agent, a surfactant, an additive in oil and gas drilling, an emulsion stabilizer, an extrusion aid, to produce vanillin, for dust control applications, etc.).

In general, lignosulfonate may be recovered by any method used to produce lignosulfonate products (e.g., provided in liquid form or as a powder). For example, lignosulfonate may be recovered using a method conventionally used for recovering lignosulfonates from waste liquor (e.g., brown or red) of a sulfite pulping process. In one embodiment, lignosulfonate is recovered by precipitation and subsequent filtering, membrane separation, amine extraction, ion exchange, dialysis, or any combination thereof.

In one embodiment, bark produced during a debarking process is recovered. For example, in one embodiment, bark produced during a debarking process is collected and combusted in a solid fuel power boiler. In one embodiment, tree tops and/or branches are collected and combusted in a solid fuel power boiler. In one embodiment, the combustion of bark and/or other otherwise unused wood products is used to boil water and produce high pressure steam (e.g., for the cogeneration of heat and power (CHP)). In one embodiment, the heat and/or electricity generated is used within the process.

To facilitate a better understanding of embodiments of the instant invention, the following examples are given. In no way should the following examples be read to limit, or define, the entire scope of the invention.

### Examples

### Example 1: Pretreatment of Softwood

Pretreatment of debarked red pine sawdust (Mesh 10) was conducted in 25 mL, stainless steel, laboratory tubular reactors (i.e., about 5 inches in length). Prior to pretreatment the red pine sawdust was air dried for about 1 week. A portion of the red pine sawdust was milled to 20 Mesh for a carbohydrate assay, which found a cellulose/glucan content of 39.7%, xylan/mannan content of 14.0%, a lignin content of 29.4%, and a total solids (TS) content of 95%, w/w on a dry basis. The carbohydrate assay was based on Determination of Structural Carbohydrates and Lignin in Biomass-LAP (Technical Report NREL/TP-510-42618).

Stock sulfurous acid solution having a SO₂ concentration between about 11.7 wt % and about 12.5 wt % (on liquor) (e.g., about 15 wt % to 16 wt % H₂SO₃ on liquor) was prepared by bubbling SO₂ into Milli-Q water cooling in an ice bath. The exact concentration of the sulfurous acid stock solution was determined using back titration with HCl (0.1M). The sulfurous acid stock solution was stored at about 4° C. Stock NaHSO₃ solutions were prepared by adding NaHSO₃ to degassed Milli-Q water and stored in filled sealed vials to eliminate headspace.

Pretreatment slurries were prepared by adding the sawdust to each laboratory tubular reactor, followed by stock NaHSO₃ solution, and a quantity of water calculated to provide the target SO₂ and alkali concentrations (e.g., based on the concentration of the stock sulfurous acid and NaHSO₃ solutions), at 10 wt % solids consistency. Once the cooled stock sulfurous acid solution was added to this mixture, the reactors were sealed immediately. Each reactor was cooked at the pretreatment temperature of 140° C., in an oil bath, for the selected pretreatment time. The pretreatment time shown includes the time for the reactor to reach the pretreatment temperature (e.g., about 5 minutes). At the end of the pretreatment, the reactors were cooled in an ice bath. All experiments conducted with or based on SO₂/sulfurous acid were carried out in a fume hood.

The concentrations and conditions used are summarized in Table 1.

TABLE 1
Pretreatment conditions

| | Run 1 | Run 2 | Run 3 |
|---|---|---|---|
| Concentration of SO₂ from | 7.8 | 7.8 | 10.5 |
| H₂SO₃ solution | | | |
| (wt %, on liquor) | | | |
| Concentration of SO₂ including | 8.4 | 8.4 | 11.1 |
| contribution from NaHSO3 | | | |
| (wt %, on liquor) | | | |
| Concentration of SO₂ including | 75.5 | 75.5 | 99.7 |
| contribution from NaHSO₃ | | | |
| (wt %, on dry weight of | | | |
| feedstock) | | | |
| Concentration of NaHSO₃ | 10 | 10 | 10 |
| (g/L) | | | |
| NaHSO₃ loading | 9 | 9 | 9 |
| (wt %, on dry weight of | | | |
| feedstock) | | | |
| Concentration of alkali (from | 0.16 | 0.16 | 0.16 |
| NaHSO₃) | | | |
| (wt %, OH, on liquor) | | | |
| Ratio of concentration of | 52.5 | 52.5 | 69.4 |
| SO2/alkali | | | |
| (where alkali is expressed as | | | |
| wt % OH) | | | |
| Pretreatment temperature (° C.) | 140 | 140 | 140 |
| Pretreatment time (min) | 120 | 120 | 180 |
| Initial pH (approx.) | 0.98-1.02 | 0.98-1.02 | 0.91 |

TABLE 2
Pretreatment results

| | Run 1 | Run 2 | Run 3 |
|---|---|---|---|
| | (8.4 wt % | (8.4 wt % | (11.1 wt % |
| | SO₂, on liq | SO₂, on liq | SO₂, on liq |
| | for 2 hours) | for 3 hours) | for 3 hours) |
| Final pH | 0.73 | 0.60 | 0.53 |
| Lignin | 91.0 | 80.6 | 83.0 |
| solubilized | | | |
| (wt %) | | | |
| Residual | 11.3 | 3.63 | 2.05 |
| hemicellulose | | | |
| (wt %) | | | |
| Hemicellulose | 72.2 | 62.98 | 49.5 |
| yield | | | |
| (wt %) | | | |

The results of the pretreatment are summarized in Table 2. The final pH refers to the pH measured after the pretreated slurry was cooled to ambient temperature. Lignin solubilized, residual hemicellulose, and hemicellulose yield were determined using a carbohydrate assay. For example, the carbohydrate content of pretreated material can be determined with a carbohydrate assay based on Determination of Structural Carbohydrates and Lignin in Biomass-LAP (Technical Report NREL/TP-510-42618). This assay can provide the cellulose content, hemicellulose content, insoluble lignin content, and soluble lignin content of the pretreated biomass, w/w on a dry basis.

The residual hemicellulose (xylan and mannan) and lignin solubilization/dissolution are calculated relative to the untreated lignocellulosic biomass. Hemicellulose yield refers to the weight percent obtained based on potential available in the feedstock. The concentration of monomeric sugars (e.g., glucose, mannose, and/or xylose) and the corresponding yields may be determined using high performance liquid chromatography (HPLC).

Referring to Table 2, the highest lignin solubilization and hemicellulose yields are obtained when the SO₂ concentration is 8.4 wt % SO₂, on liquor, and the pretreatment is conducted for 2 hours. At the longer times and/or higher acid concentrations, the hemicellulose yield begins to decrease, less lignin is solubilized, and/or lignin begins to condense.

### Example 2: Enzymatic Hydrolysis

Washed pretreatment samples were prepared by suspending a portion of pretreated sample in ultra-purified water (Milli-Q™), filtering the suspension through glass fiber filter paper (G6, 1.6 microns), and then repeating.

The washed pretreatment solids were hydrolyzed in 50 mL Erlenmeyer flasks, at a consistency of 15 wt %, with sodium citrate (1 M of citrate buffer pH added to a final concentration of 0.1M). The flasks were incubated at 52° C., with moderate shaking at about 250 rpm, for 30 minutes to equilibrate substrate temperature.

Hydrolysis was initiated by adding liquid cellulase enzyme. Enzyme was added at a dosage of 2.5-9 mg/g (i.e., mg protein/g of cellulose). The flasks were incubated at 52° C. in an orbital shaker (250 rpm) for various hydrolysis times (e.g., 200 hours).

The hydrolyses were followed by measuring the sugar monomers in the hydrolysate. More specifically, aliquots obtained at various hours of hydrolysis, were used to analyze the sugar content. More specifically, HPLC was used to measure the amount of glucose, which was used to determine the cellulose conversion. The cellulose conversion, which is expressed as the amount of glucose released during enzymatic hydrolysis of the solid fraction, and thus may be referred to as glucose conversion herein, was determined using the following:

Cellulose conversion=concentration of glucose in aliquot/maximum glucose concentration at 100% conversion.

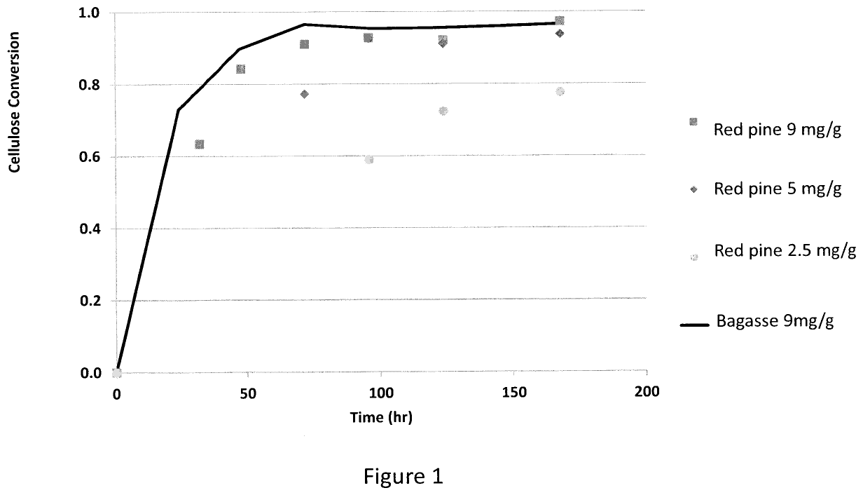

FIGS. 1 to 3 show plots of cellulose conversion for the washed solids from Runs 1 to 3, respectively (e.g., see Table 1), as compared to the cellulose conversion of bagasse pretreated under substantially the same pretreatment conditions. For example, for comparative purposes, the hydrolyses results are compared to those of bagasse pretreated at 140° C., for 2-3 hours, with a SO₂ concentration between 8.4 wt % and 11.1 wt %, on liquor, an alkali concentration of about 0.16 wt %, OH, on liquor, and a solids consistency of 10 wt %.

FIG. 1 shows the cellulose conversion for the washed solids from Run 1 (e.g., a pretreatment temperature of 140° C., a pretreatment time of 2 hours, a SO₂ concentration of 8.4 wt % (on liquor), an alkali concentration of about 0.16 wt %, (OH, on liquor), and a solids consistency of 10 wt %). The cellulose conversion plots are provided for enzyme loadings of 2.5 mg/g, 5 mg/g, and 9 mg/g. The cellulose conversion was not measured at early conversion times when the enzyme dosage is low due to the high consistency.

Referring to FIG. 1, these pretreatment conditions permitted more than 85% cellulose conversion for the enzymatic hydrolyses at the two higher enzyme doses, and began to approach the results achieved for bagasse (e.g., both at 9 mg/g enzyme). This is remarkable because softwood is generally considered to be one of the most difficult lignocellulosic feedstocks to enzymatically hydrolyze to glucose, and it has now been demonstrated that these pretreatment conditions can be used to provide a good pretreatment for both bagasse and resinous softwood.

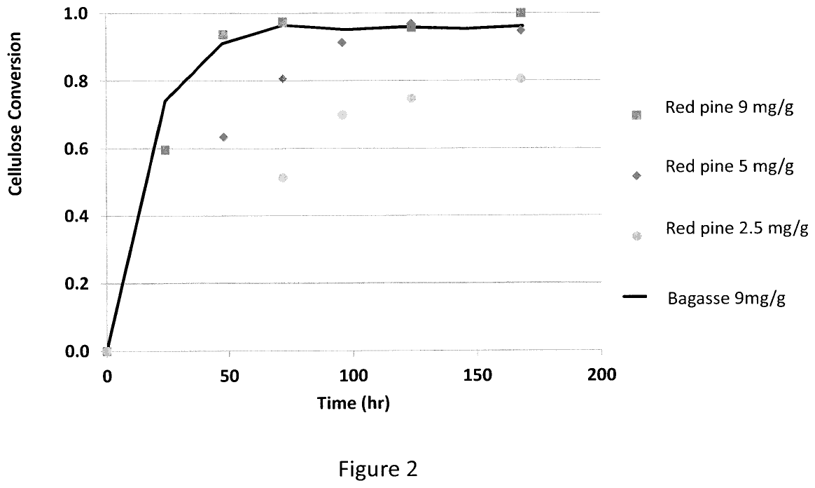

FIG. 2 shows the cellulose conversion for the washed solids from Run 2 (e.g., a pretreatment temperature of 140° C., a pretreatment time of 3 hours, a SO₂ concentration of 8.4 wt % (on liquor), an alkali concentration of about 0.16 wt %, (OH, on liquor), and a solids consistency of 10 wt %). The cellulose conversion plots are provided for enzyme loadings of 2.5 mg/g, 5 mg/g, and 9 mg/g. The cellulose conversion was not measured at early conversion times when the enzyme dosage is low due to the high consistency.

Referring to FIG. 2, increasing the pretreatment time from 2 hours to 3 hours allows the enzymatic hydrolysis with a 9 mg/g dose to reach about 100% conversion, and the hydrolysis with a 2.5 mg/g dose of enzyme to reach 80% conversion. Moreover, the cellulose conversion for the enzymatic hydrolysis of softwood may be higher than that obtained for bagasse.

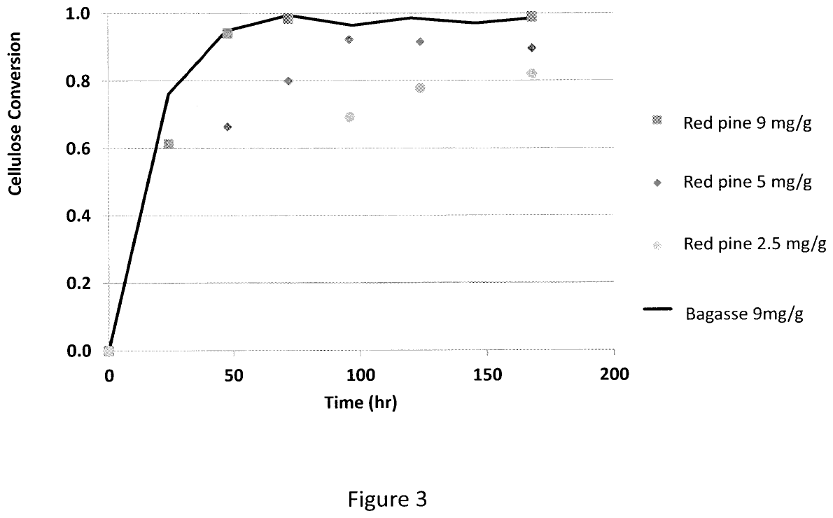

FIG. 3 shows the cellulose conversion for the washed solids from Run 3 (e.g., a pretreatment temperature of 140° C., a pretreatment time of 3 hours, a SO₂ concentration of 10.5 wt % (on liquor), an alkali concentration of about 0.16 wt %, (OH, on liquor), and a solids consistency of 10 wt %). The cellulose conversion plots are provided for enzyme loadings of 2.5 mg/g, 5 mg/g, and 9 mg/g. The cellulose conversion was not measured at early conversion times when the enzyme dosage is low due to the high consistency.

Referring to FIG. 3, increasing the SO₂ concentration from 8.4 to 11.1 wt % (on liquor) allows the enzymatic hydrolysis with a 9 mg/g dose to reach about 100% conversion, and the hydrolysis with a 2.5 mg/g dose of enzyme to reach more than 80% conversion. Moreover, the cellulose conversion for the enzymatic hydrolysis of softwood may be higher than that obtained for bagasse.

The enzymatic hydrolysis results from Runs 2 and 3 are notable for at least two reasons. First, a cellulose conversion of 100% is very good, especially for softwood. Second, this high cellulose conversion was obtained even though the final pH of the pretreatment was less than 0.7. Such low pH values are typically associated with lignin condensation, which is believed to have a role in inhibition of the enzymes used in the hydrolysis reaction. As discussed above, lignin condensation is particularly problematic during the acid sulfite pulping of resinous softwood. However, for Runs 2 and 3, the enzymatic hydrolysis results for red pine are very good even though the final pH was quite low. Without being bound by theory, these surprisingly good hydrolysis results may be related to a relatively high SO₂ loading on dry solids (e.g., greater than 75 wt %), the relatively high SO₂ concentration in the pretreatment liquor (e.g., greater than 8.4 wt %), the formation of significant amounts of lignosulfonic acid (LSA), the relatively low temperature (e.g., about 140° C.), and/or a relatively high SO₂/alkali concentration ratio (e.g., greater than about 52%, where the alkali concentration is expressed as weight percent hydroxide). For example, it may be advantageous to provide a relatively high SO₂ loading on dry solids (e.g., greater than 36 wt %) with a relatively low alkali loading (e.g., less than 0.25 wt % expressed as weight percent hydroxide on liquor).

In any case, the relatively low pH values may provide the low residual hemicellulose levels (e.g., ˜2 wt % to about ˜11 wt %). In general, there is often a tradeoff between decreasing residual hemicellulose levels and increasing lignin solubilization. However, for Run 3 the pretreatment solubilized about 98 wt % of the hemicellulose and about 83 wt % of the lignin. Accordingly, in addition to improving the enzymatic hydrolysis, these pretreatment conditions may improve the yield of products from the non-cellulose fraction of the softwood. Advantageously, the pretreatment does not rely on adding an organic solvent to the pretreatment. Further advantageously, the pretreatment can be conducted in a single stage (e.g., separate stages that promote lignin and hemicellulose dissolution are not required).

Of course, the above embodiments have been provided as examples only. It will be appreciated by those of ordinary skill in the art that various modifications, alternate configurations, and/or equivalents will be employed without departing from the spirit and scope of the invention. Accordingly, the scope of the invention is therefore intended to be limited solely by the scope of the appended claims.

1. A process for producing a fuel from softwood, said process comprising:
(a) obtaining a feedstock comprising softwood;
(b) pretreating the feedstock, said pretreating comprising heating the feedstock in a pretreatment liquor comprising sulfur dioxide and bisulfite salt, said heating conducted between 110° C. and 160° C., wherein the pretreatment liquor has a pH at 25° C. that is less than 1.3 and has a sulfur dioxide concentration that is greater than 6.5 wt % on liquor,
(c) obtaining a slurry of pretreated material produced in (a), said slurry having a solid fraction comprising cellulose and a liquid fraction comprising solubilized hemicellulose;
(d) hydrolyzing the cellulose to glucose, said hydrolyzing comprising adding cellulase to at least the solid fraction;
(e) fermenting the glucose to a fermentation product, said fermenting comprising adding a microorganism to at least the glucose; and
(f) recovering the fermentation product,
wherein the fuel comprises the fermentation product.

2. The process according to claim 1, wherein the softwood comprises resinous softwood.

3. The process according to claim 1, wherein the softwood comprises pine, Douglas fir, or a combination thereof.

4. The process according to claim 1, wherein the feedstock comprises woodchips.

5. The process according to claim 1, wherein the feedstock comprises sawdust.

6. The process according to claim 1, wherein said heating is conducted between 120° C. and 150° C.

7. The process according to claim 1, wherein said heating is conducted for between 30 minutes and 4 hours.

8. The process according to claim 1, wherein the liquid to solid weight ratio in the pretreatment is between 1.5 and 9.

9. The process according to claim 1, wherein the concentration of sulfur dioxide in the pretreatment is between 7.8 wt % on liquor and 19.5 wt % on liquor.

10. The process according to claim 1, wherein the concentration of sulfur dioxide in the pretreatment is greater than 65 wt % on dry solids.

11. The process according to claim 1, wherein a concentration of alkali in the pretreatment is at least 0.05 wt % expressed as weight percent hydroxide on liquor.

12. The process according to claim 1, wherein a ratio of concentration of sulfur dioxide on liquor to concentration of alkali, expressed as weight percent hydroxide, on liquor is greater than 30.

13. The process according to claim 1, wherein a concentration of sulfur dioxide in the pretreatment is greater than 36 wt % on dry solids, and wherein a concentration of alkali is less than 0.25 wt % expressed as weight percent hydroxide on liquor.

14. The process according to claim 1, wherein the pH of the pretreatment liquor at 25° C. is between 0.9 and 1.1.

15. The process according to claim 1, wherein the pH of the slurry of pretreated material is less than 1.

16. The process according to claim 1, comprising:
subjecting the slurry of pretreated material to a solid-liquid separation to separate the solid fraction and the liquid fraction; and
washing the solid fraction produced by the solid-liquid separation with water,
wherein adding cellulase to at least the solid fraction comprises adding the cellulase to the washed solid fraction.

17. The process according to claim 1, comprising producing one or more products from the liquid fraction, said one or more products comprising at least one of xylose, xylitol, methane, ethanol, or lignosulfonate.

18. The process according to claim 1, wherein the fermentation product is ethanol.

19. A process for producing ethanol comprising:
(a) obtaining a feedstock, said feedstock comprising softwood woodchips;
(b) pretreating the feedstock, said pretreating comprising heating the feedstock in a pretreatment liquor comprising sulfur dioxide and bisulfite salt, said heating conducted between 110° C. and 160° C. for at least 30 minutes, wherein the pretreatment liquor has a pH at 25° C. that is less than 1.3 and has a sulfur dioxide concentration that is greater than 6.5 wt % on liquor;
(c) obtaining a slurry of pretreated material produced in (a), said slurry having a solid fraction comprising cellulose and a liquid fraction comprising solubilized hemicellulose;
(d) hydrolyzing the cellulose to glucose, said hydrolyzing comprising adding cellulase to at least the solid fraction;
(e) fermenting the glucose to ethanol, said fermenting comprising adding a microorganism to at least the glucose;
(f) recovering the ethanol; and
(g) producing one or more products from the liquid fraction, said one or more products comprising at least one of xylose, xylitol, methane, ethanol, or lignosulfonate.
